(19) United States
(12) Patent Application Publication
(10) Pub. No.: US 2010/0047279 A1
(43) Pub. Date: Feb. 25, 2010

(54) IMMUNOLOGICAL COMPOSITION

(75) Inventors: Philip Ralph LEHRBACH, Wahroonga (AU); William John CHESHIRE, Emu Plains (AU); Zhixian Xin, Doonside (AU)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/543,536

(22) Filed: Aug. 19, 2009

Related U.S. Application Data
(60) Provisional application No. 61/092,091, filed on Aug. 27, 2008.

(30) Foreign Application Priority Data
Aug. 19, 2008 (AU) 2008904261

(51) Int. Cl. A61K 39/12; A61K 39/15; A61K 39/205; A61K 39/245; A61K 39/02; A61K 47/00

(52) U.S. Cl. 424/215.1; 424/204.1; 424/224.1; 424/229.1; 424/234.1; 424/278.1

(57) ABSTRACT

The present invention relates to immunological compositions comprising sulpholipo-cyclodextrin (SL-CD) and, saponin or Quil A, and optionally at least one antigen. The invention relates to methods and immunological compositions comprising at least one antigen, which may be a veterinary antigen. The veterinary antigen in the methods and immunological compositions of the invention may be a bovine antigen. The invention relates to methods and immunological compositions comprising bovine ephemeral fever virus (BEFV), bovine herpesvirus 1 (IBR) or bluetongue virus (BTV). The invention comprises methods for eliciting an immune response against BEFV, IBR, or BTV in an animal, which comprises administering to the animal a composition of the invention. In the invention, particularly the immune response is a protective immune response. The invention comprises a method for preparing an immunological composition comprising adding Quil A to a virus.

### CROSS-REFERENCE TO RELATED APPLICATIONS

This application claims the benefit under 35 U.S.C. §119(a) of Australian Provisional Application No. 2008904261 filed Aug. 19, 2008 and under 35 U.S.C. §119(e) of U.S. Provisional Application No. 61/092,091, filed Aug. 27, 2008. The entire contents of these applications are incorporated herein by reference.

### FIELD OF THE INVENTION

The present invention relates to immunological compositions comprising sulpholipo-cyclodextrin (SL-CD), and saponin or Quil A, and optionally at least one antigen. The invention also relates to methods for preparing immunological compositions comprising SL-CD, saponin or Quil A, and an antigen. The present invention also provides methods for using immunogenic compositions for eliciting an immune response to bovine ephemeral fever virus (BEFV), to bovine herpesvirus 1 (IBR), or to bluetongue virus (BTV). The present invention provides kits comprising an immunological composition of the invention.

### BACKGROUND OF THE INVENTION

Saponin adjuvants are a known class of adjuvants that have been used commercially in vaccines for animals. Saponins are a class of secondary metabolites found in various plant species. They are amphipathic glycosides grouped phenomenologically by the soap-like foaming they produce when shaken in aqueous solutions. Structurally, saponins are composed of one or more hydrophilic glycoside moieties combined with a lipophilic triterpene derivative. Commercial saponins are mainly isolated from the bark of the South American tree Quillaja Saponaria Molina and the Mohave Yucca plant which is also called Yucca schidigera. Saponins are available from several sources including Berghausen Corporation (Cincinnati, Ohio). The refined form of saponin is commonly referred to as Quil A and is commercially available from several sources including Berghausen Corporation, Sergeant Chemical Company (Clifton, N.J.), Superfos a/s (Vedbaek, Denmark), and Brenntag Biosector (Frederikssund, Denmark). The physical and chemical characteristics of Quil-A are set out in the trade literature available from Superfos, entitled Purified Saponin Adjuvant Quil-A. Quil-A is characterised chemically as a carbohydrate moiety in glycosidic linkage to the triterpenoid quillaic acid.

Several U.S. patents have issued discussing Quil A as an adjuvant. For example, U.S. Pat. Nos. 6,416,764 and 6,291,228 relate to vaccines using Quil A as an adjuvant and comprising a non-cytopathogenic strain of bovine viral diarrhea virus. U.S. Pat. No. 4,432,969 relates to an inhalant allergen composition comprising an inhalant allergen and a saponin or Quil A adjuvant.

U.S. Pat. No. 4,900,549 describes a process for preparing immunogenic complexes containing Quil A.

U.S. Pat. No. 6,165,995 relates to the preparation of SL-CD derivatives. U.S. Pat. No. 6,610,310 is related to polyionic polymers, such as SL-CD, as adjuvants.

Bovine Ephemeral Fever (BEF) is a debilitating viral disease affecting both dairy and beef cattle, particularly in northern Australia. BEF is also recognized in most Asian countries where cattle are commercially raised. BEF is known as “three day sickness” and can substantially affect dairy milk yield or cause morbidity in beef and dairy cattle (Walker, P. J., 2005, Curr. Top. Microbiol. Immunol. 292: 57-80).

The causative agent of BEF is bovine ephemeral fever virus (BEFV). This virus is a rhabdovirus that has been classified in the genus Ephemerovirus. The BEFV virion is bullet or cone shaped and contains a negative single-strand RNA genome. The BEFV genome encodes a nucleoprotein, a polymerase-associated protein, a matrix protein, a large RNA-dependent RNA polymerase, and two glycoproteins.

A modified live BEF vaccine has been available in Australia for many years and is given by annual booster before the BEF season. This vaccine requires a veterinary prescription and is presented in a freeze-dried form requiring reconstitution with a diluent containing adjuvant before administration. Naïve animals require two vaccine doses followed by annual revaccination.

PCT Publication No. WO/1994004685 relates to the preparation of a BEFV vaccine comprising the BEFV surface glycoprotein.

Vanselow et al. (1995, Vet. Microbiol. 46:117-130) describes testing of various BEF vaccines.

Hsieh et al. (2006, J. Vet. Med. Sci. 68: 543-548) relates to BEFV vaccines using viral strains Tn88128 and Tn73. These vaccines were prepared by inactivating the virus by the addition of binary ethylimine and aluminum hydroxide or water:oil:water adjuvants.

A recombinant vaccine comprising the BEFV structural glycoprotein cloned into a lumpy skin disease virus (type SA-Neethling) vector has been described by Wallace, D. B. and Viljoen G. J. (2005, Vaccine 23:3061-3067).

Chuang et al. (2007, J. Virol. Meth. 145:84-87) relates to using RNA interference and suppression of BEFV surface glycoprotein gene expression.

Bovine herpesvirus-1 is also known as infectious bovine rhinotracheitis virus. It is found abbreviated as either BHV or IBR. Bovine herpesvirus-1 is a virus of the family Herpesviridae that causes diseases in cattle, including rhinotracheitis, vaginitis, balanoposthitis, abortion, conjunctivitis, and enteritis. BHV-1 is also a contributing factor in shipping fever. It is spread through sexual contact, artificial insemination, and aerosol transmission. Like other herpesviruses, BHV-1 causes a lifelong latent infection and shedding of the virus. There is a vaccine available which reduces the severity and incidence of disease. The respiratory disease caused by BHV-1 is commonly known as infectious bovine rhinotracheitis.

Bluetongue virus (BTV) is the prototype virus of the genus Orbivirus, which belongs to the double-stranded RNA family Reoviridae. BTV causes serious disease in livestock such as sheep, goats, cattle and deer. Twenty-four serotypes are reported in the literature as causing problems ranging from unapparent infection to acute fulminating infection. Chronic, persistent virus shedding cattle have also been recognized. Vaccines are available for the treatment of Bluetongue in livestock.

### SUMMARY OF THE INVENTION

The present disclosure provides immunological compositions comprising sulpholipo-cyclodextrin (SL-CD) and saponin, and optionally, at least one antigen. In some embodiments of the invention the saponin is Quil A. In some embodiments of the invention, the at least one antigen is selected from bacteria, viruses, peptides, polypeptides, nucleic acids, or a combination thereof. In some embodiments of the invention, the at least one antigen is a veterinary antigen. In some embodiments of the invention, the veterinary antigen is a bovine antigen. In some embodiments of the invention, the antigen is a viral antigen. In some embodiments of the invention the viral antigen is bovine herpesvirus 1 (IBR), bluetongue virus (BTV), or bovine ephemeral fever virus (BEFV). The viral antigen can be live-attenuated, recombinant, killed, or inactivated. In some embodiments, the invention provides an immunogenic composition where the antigen is a live-attenuated virus. In some embodiments of the invention, the virus is bovine ephemeral fever virus (BEFV). In various embodiments of the invention, the virus is from a frozen stock, a dried stock, a freeze-dried stock, or a fresh stock. In various embodiments saponin is present in the immunological composition of the invention at a final concentration of about 0.5 mg/mL. In various embodiments, Quil A is present in the immunological composition of the invention at a final concentration of about 0.1 mg/mL to about 0.2 mg/mL. In some embodiments, in the immunological composition of the invention Quil A is present at a final concentration of about 0.158 mg/mL. In various embodiments, in the immunological composition of the invention SL-CD is present at a final concentration of about 0.2 mL/mL. In some embodiments, the immunological composition of the invention comprising saponin and SL-CD or comprising Quil A and SL-CD, comprises at least one additional adjuvant. In various embodiments of the invention, the additional adjuvant is selected from aluminum hydroxide, SP-oil, or carbopol. In some embodiments of the invention, the antigen is a polypeptide, which in some embodiments is a viral subunit. In some embodiments of the invention, the viral subunit is selected from BEFV, IBR, or BTV.

In one embodiment, the present invention provides a method for eliciting an immune response against a veterinary antigen in an animal, which comprises administering to the animal an immunogenic composition comprising saponin, SL-CD, and at least one veterinary antigen. In one embodiment, the immune response is elicited after administration of a single dose of the immunogenic composition. In one embodiment, the present invention provides a method for eliciting an immune response against IBR in an animal, which comprises administering to the animal an immunogenic composition comprising saponin, SL-CD, and at least IBR as an antigen. In one embodiment, the present invention provides a method for eliciting an immune response against BTV in an animal, which comprises administering to the animal an immunogenic composition comprising saponin, SL-CD, and at least BTV as an antigen.

In one embodiment, the present invention provides a method for eliciting an immune response against BEFV in an animal, which comprises administering to the animal an immunogenic composition comprising Quil A, SL-CD, and an antigen. In one embodiment, the immune response is elicited after administration of a single dose of the immunogenic composition. In one embodiment, the present invention provides a method for eliciting an immune response against IBR in an animal, which comprises administering to the animal an immunogenic composition comprising saponin, SL-CD, and an antigen. In one embodiment, the present invention provides a method for eliciting an immune response against BTV in an animal, which comprises administering to the animal an immunogenic composition comprising saponin, SL-CD, and an antigen. In some embodiments of the invention, the immune response elicited after administration of the immunogenic composition of the invention is a protective immune response.

In one embodiment, the present invention provides an immunogenic composition prepared by combining Quil A and virus prior to adding SL-CD. The virus can be live-attenuated, recombinant, killed, or inactivated. In some embodiments of the invention, the Quil A and the virus are combined at room temperature. In some embodiments of the invention, the Quil A and the virus are combined for at least 15 minutes. In some embodiments of the invention, the Quil A and the virus are combined for at least 120 minutes.

In one embodiment, the present invention provides a kit comprising an immunogenic composition of the invention for eliciting an immune response in an animal.

In various embodiments, the present invention provides methods for inducing an immune response in cattle against viral infection or bovine ephemeral fever caused by BEFV. The methods for inducing an immune response against BEFV comprise administering to the cattle a composition comprising BEFV, SL-CD, and Quil A.

In one embodiment, the present invention provides an immunogenic composition or vaccine comprising an immunologically effective amount of BEFV, SL-CD, and Quil A.

In various embodiments, the present invention provides methods for inducing in cattle an immune response against herpesviral infection or bovine rhinotracheitis caused by IBR. The methods for inducing an immune response against IBR comprise administering to the cattle a composition comprising IBR, SL-CD, and saponin.

In various embodiments, the present invention provides methods for inducing in cattle an immune response against viral infection or bovine bluetongue caused by BTV. The methods for inducing an immune response against BTV comprise administering to the cattle a composition comprising BTV, SL-CD, and saponin.

In one embodiment, the present invention provides an immunogenic composition or vaccine comprising an immunologically effective amount of IBR, SL-CD, and saponin.

In one embodiment, the present invention provides an immunogenic composition or vaccine comprising an immunologically effective amount of BTV, SL-CD, and saponin.

### DETAILED DESCRIPTION OF THE INVENTION

The present disclosure is based, in part, upon the discovery that an immunological composition comprising SL-CD, and saponin or Quil A, can enhance the immunogenicity of at least one antigen. In various embodiments of the invention, the at least one antigen may be selected from bacteria, viruses, peptides, polypeptides, nucleic acids, or combinations thereof.

In some embodiments of the invention the at least one antigen is a veterinary antigen. In various embodiments of the invention a veterinary antigen may be a bovine antigen.

In one embodiment of the invention, the veterinary antigen may be a viral antigen. In some embodiments of the invention, the viral antigen includes but is not limited to a strain of BEFV, IBR, or BTV. BEFV is a rhabdovirus known to cause bovine ephemeral fever in Australia, Africa, the Middle East, and Asia. Many strains of BEFV are known, such as, BB2271-919 and its parent strain (919), TN73, Tn88128, strains 1-11 of BEFV2001, or strains 1-3 of BEFV2004. Selection of the strain can be varied depending on the Country where the immunogenic composition of the invention is to be used. Bovine herpesvirus-1 is also referred to as BHV or IBR, and is a virus of the Herpesviridae family that causes diseases in cattle, including rhinotracheitis, vaginitis, balanoposthitis, abortion, conjunctivitis, and enteritis. IBR is also a contributing factor in shipping fever. It is spread through sexual contact, artificial insemination, and aerosol transmission. Like other herpesviruses, IBR causes a lifelong latent infection and shedding of the virus. The respiratory disease caused by IBR is commonly known as infectious bovine rhinotracheitis.

Bluetongue virus (BTV) is the prototype virus of the genus Orbivirus, which belongs to the double-stranded RNA family Reoviridae. BTV causes serious disease in livestock such as sheep, goats, cattle and deer. Twenty-four serotypes are reported in the literature as causing problems ranging from unapparent infection to acute fulminating infection. Chronic, persistent virus shedding cattle have also been recognized. Bluetongue has been observed in Australia, North America, Africa, the Middle East, Asia, and Europe.

In various embodiments of the invention, the virus in the immunogenic composition can be live-attenuated, recombinant, killed, or inactivated. Methods for preparing live-attenuated virus are known in the literature. For example, a virus may be attenuated via passage of the virus through a foreign host such as tissue culture, embryonated eggs, or live animals. Attenuated BEFV may be selected for preferential growth in non-bovine cells and, in the course of selection, become less able to grow in bovine cells. Because these attenuated strains replicate poorly in bovine hosts, they induce immunity but not disease when given to cattle. A virus is said to be attenuated if it has decreased virulence for the native host and increased its virulence for the new host. Some attenuated virus strains may occur naturally. Genetic engineering may be used to attenuate viruses in defined ways. Methods of preparing a killed or inactivated virus for use in immunogenic compositions, vaccines, and methods are known in the art. In a chemical inactivation process, a suitable virus sample, or serum sample containing the virus, is treated for a sufficient length of time with a sufficient amount or concentration of inactivating agent at a sufficiently high (or low, depending on the inactivating agent) temperature or pH to inactivate the virus. For example, a virus may be treated with inactivating agents such as formalin, binary ethyleneimine (BEI), or hydrophobic solvents, acids, etc. The virus may be inactivated by irradiation with ultraviolet light or X-rays, by heating, etc. Inactivation by heating is conducted at a temperature and for a length of time sufficient to inactivate the virus. Inactivation by irradiation is conducted using a wavelength of light or other energy source for a length of time sufficient to inactivate the virus. In some embodiments of the invention, the immunogenic composition comprises a live-attenuated virus.

In some embodiments of the invention, the immunogenic composition comprises an antigen obtained from a frozen stock, a dried stock, or a fresh stock. If the antigen is obtained from a dried stock, it may be obtained from a freeze-dried stock. In some embodiments, the immunogenic composition of the invention comprises an antigen from a frozen stock.

Saponins are steroid or triterpene glycosides widely distributed in the plant and marine animal kingdoms. Saponins are noted for forming colloidal solutions in water, which foam on shaking, and for precipitating cholesterol. When saponins are near cell membranes they create pore-like structures in the membrane, which cause the membrane to burst. Saponins are used as adjuvants in vaccines for animals. The adjuvant and haemolytic activity of individual saponins has been extensively studied (Lacaille-Dubois and Wagner, 1996, A review of the biological and pharmacological activities of saponins” Phytomedicine vol 2 pp 363-386). Saponins are a class of secondary metabolites found in various plant species. They are amphipathic glycosides grouped phenomenologically by the soap-like foaming they produce when shaken in aqueous solutions. Structurally they are composed of one or more hydrophilic glycoside moieties combined with a lipophilic triterpene derivative. Commercial saponins are mainly extracted from Quillaja saponaria Molina and Yucca schidigera. “Quil A” refers to the refined form of saponin.

Saponin may be present in the immunogenic compositions of the invention at a final concentration of about 0.4 mg/mL to about 0.6 mg/mL. Saponin may be present in the immunogenic compositions of the invention at a final concentration of about 0.5 mg/mL. Quil A may be present in the immunogenic compositions of the invention at a final concentration of about 0.1 mg/mL to about 0.2 mg/mL. Quil A may be present in the immunogenic compositions of the invention at a final concentration of about 0.12 mg/mL to about 0.18 mg/mL. Quil A may be present in the immunogenic compositions of the invention at a final concentration of about 0.14 mg/mL to about 0.16 mg/mL. Quil A may be present in the immunogenic compositions of the invention at a final concentration of about 0.158 mg/mL. In one embodiment, Quil A is present at a final concentration of about 0.158 mg/mL in the immunogenic compositions of the invention.

Sulfolipo-cyclodextrin in squalane in-water emulsion (SL-CD/squalane) was used in the preparation of the different vaccines. SL-CD/squalene may be prepared as described by Hilgers et al. (Sulfolipo-cyclodextrin in squalane in-water as a novel and safe vaccine adjuvant. Vaccine 17 (1999), pp. 219-228; Fort Dodge Animal Health Holland, Weesp, The Netherlands)

SL-CD may be present in the immunogenic compositions of the invention at a final concentration of about 0.09 mL/mL to about 0.3 mL/mL. SL-CD may be present in the immunogenic compositions of the invention at a final concentration of about 0.1 mL/mL, at about 0.15 mL/mL, at about 0.17 mL/mL, at about 0.2 mL/mL, or at about 0.25 mL/mL. The immunogenic compositions of the invention may further comprise at least one adjuvant in addition to Quil A and SL-CD. Such additional adjuvant may be selected from any one of the adjuvants known in the art, as discussed in further detail herein.

In some embodiments, the invention provides a method for eliciting an immune response, which comprises administering to an animal an immunological composition comprising saponin and SL-CD. In some embodiments, the method comprises administering to an animal an immunological composition comprising saponin, SL-CD, and at least one antigen. In some embodiments, the at least one antigen is a veterinary antigen. In some embodiments of the invention the veterinary antigen is a bovine antigen. In some embodiments of the invention the veterinary antigen is a viral antigen. In some embodiments, the viral antigen is BEHV, IBR or BTV. In some embodiments, the virus is live-attenuated, recombinant, killed, or inactivated. In some embodiments, the virus is killed. In some embodiments, the antigen is obtained from a frozen stock, a dried stock, or a fresh stock. If the antigen is obtained from a dried stock, it may be obtained from a freeze-dried stock. In some embodiments, the antigen is obtained from a frozen stock.

In some embodiments, the invention provides a method for eliciting an immune response, which comprises administering to an animal an immunological composition comprising Quil A and SL-CD. In some embodiments, the method comprises administering to an animal an immunological composition comprising Quil A, SL-CD, and at least one antigen. In some embodiments, the at least one antigen used in the method is a veterinary antigen. In some embodiments, the veterinary antigen used the method is a bovine antigen. In some embodiments, the antigen is a viral antigen. In some embodiments, the viral antigen is IBR, BEFV, or IBR. In some embodiments, the viral antigen is live-attenuated, recombinant, killed, or inactivated. In some embodiments, the virus is live-attenuated. In some embodiments, the antigen is obtained from a frozen stock, a dried stock, or a fresh stock. If the antigen is obtained from a dried stock, it may be obtained from a freeze-dried stock. In some embodiments, the antigen is obtained from a frozen stock.

The immunogenic compositions of the invention may elicit an immune response after the administration of multiple doses. In some embodiments, the immunogenic compositions of the invention may elicit an immune response after the administration of two doses. In some embodiments, the immunogenic compositions of the invention elicit an immune response after the administration of a single dose. The immune response elicited by the immunogenic compositions of the invention may be a protective immune response. After administration of an initial dose of the immunogenic composition of the invention, a booster dose may be administered after a period of about four weeks to enhance the immunogenic response. Further booster dosages may also be administered.

In one embodiment, the invention provides a kit for eliciting an immune response in an animal. In some embodiments, the kit comprises an immunogenic composition comprising saponin and SL-CD and, optionally, at least one antigen. In some embodiments, the saponin in the kit is Quil A. In some embodiments, the at least one antigen in the kit is a veterinary antigen. In some embodiments, the veterinary antigen in the kit is a bovine antigen. In some embodiments, the veterinary antigen in the kit is a viral antigen. In some embodiments, the viral antigen in the kit is BEFV, IBR, or BTV. In some embodiments, the antigen in the kit may be live-attenuated, recombinant, killed or inactivated. In some embodiments, the antigen in the kit is live-attenuated. In some embodiments, the antigen in the kit is killed. In some embodiments, the antigen in the kit may be obtained from a frozen stock, dried stock, or a fresh stock. In some embodiments, the antigen in the kit is obtained from a frozen stock.

The invention provides kits, immunological compositions, and vaccines comprising SL-CD and, saponin and/or Quil A, which can comprise at least one additional adjuvant. Among the adjuvants which may be used, there may be mentioned by way of example aluminium hydroxide, pyridine, dimethyldioctadecylammonium bromide (also known as DDAB or DODAB), polyphosphazenes, oil-in-water emulsions based on mineral oil such as SPT emulsion (see, for example Vaccine Design, The Subunit and Adjuvant Approach, 1995, edited by Michael F. Powel and Mark J. Newman, Plenum Press, New York and London, pages 147-204), water-in-oil emulsions based on metabolizable oil as described in U.S. Pat. No. 6,368,601, as well as the emulsions described in U.S. Pat. No. 5,422,109. Other examples of suitable adjuvants include squalane and squalene (or other oils of animal origin); block copolymers such as Pluronic® (L121) saponin; detergents such as Tween®-80, mineral oils such as DRAKEOL®, or Marcol®; vegetable oils such as peanut oil; corynebacterium-derived adjuvants such as corynebacterium parvum; propionibacterium-derived adjuvants; Mycobacterium bovis (Bacillus Calmette and Guerinn, or BCG); interleukins such as interleukin 2 and interleukin-12; monokines such as interleukin 1; tumor necrosis factor; interferons such as gamma interferon; liposomes; iscom adjuvant; mycobacterial cell wall extract; synthetic glycopeptides such as muramyl dipeptides or other derivatives; Avridine; Lipid A; dextran sulfate; DEAE-Dextran or DEAE-Dextran with aluminum phosphate; carboxypolymethylene, such as Carbopol®; EMA; acrylic copolymer emulsions such as Neocryl® A640 (see U.S. Pat. No. 5,047,238); vaccinia or animal poxvirus proteins; subviral particle adjuvants such as orbivirus; cholera toxin; dimethyldiocledecylammonium bromide; or mixtures thereof. Other adjuvants may be selected from surfactants (e.g., hexadecylamine, octadecylamine, lysolecithin, dimethyldioctadecylammonium bromide, N,N-dioctadecyl-n′-N-bis(2-hydroxyethylpropane di-amine), methoxyhexadecylglycerol, and pluronic polyols); polyanions (e.g., pyran, dextran sulfate, poly IC, polyacrylicacid, carbopol), peptides (e.g., muramyl dipeptide, dimethylglycine, tuftsin), oil emulsions, alum, and mixtures thereof. It is also possible to choose combinations of adjuvants.

In one embodiment, the invention provides an immunogenic composition prepared by combining Quil A and the antigen prior to adding an additional antigen such as SL-CD. It is understood by those skilled in the art that combining the virus with Quil A will lower the effective virus titer (Walker, P. J., 2005, Curr. Top. Microbiol. Immunol. 292: 57-80). Combination of the Quil A and the antigen, prior to adding at least one other ingredient to the immunogenic composition, may be performed for any length of time. One of skill in the art will readily understand that the immunogenic composition of the invention may be prepared by combining the Quil A and the antigen, prior to adding at least one other ingredient to the immunogenic composition, for various periods of time. For example, the Quil A and the antigen may be combined from at least 5 minutes to at least 200 minutes. In some embodiments, the Quil A and the antigen are combined for any length of time including from at least 10 minutes to at least 190 minutes. In some embodiments, the Quil A and the antigen are combined for at least 15 minutes. It is understood by those of skill in the art that the immunogenic composition of the invention may be prepared by combining the Quil A and the antigen at any one of many temperatures. Combination of the Quil A and the antigen may be performed at temperatures lower or higher than room temperature as long as the resulting composition is immunogenic. The Quil A and the antigen may be combined at room temperature. In some embodiments, the antigen in the immunogenic composition prepared by combining Quil A and an antigen prior to adding SL-CD is a virus. In some embodiments, the virus is BEFV. In some embodiments, the virus may be live-attenuated, recombinant, killed or inactivated. In some embodiments, the antigen is live-attenuated. In some embodiments, the antigen may be obtained from a frozen stock, dried stock, or a fresh stock. In some embodiments, the antigen is obtained from a frozen stock.

In one embodiment, the present invention provides an immunogenic composition for eliciting an immune response, the immunogenic composition comprising saponin and SL-CD. In some embodiments of the invention, the immunogenic composition for eliciting an immune response comprises saponin and SL-CD; and a least one antigen. In some embodiments of the invention, the saponin in the immunogenic composition for eliciting an immune response is Quil A. In some embodiments of the invention the at least one antigen in the immunogenic composition for eliciting an immune response may be selected from bacteria, viruses, peptides, polypeptides, nucleic acids, or combinations thereof. In some embodiments, in the immunogenic composition of the invention, the at least one antigen is a veterinary antigen. In some embodiments, in the immunogenic composition of the invention, the veterinary antigen is a bovine antigen. In some embodiments, in the immunogenic composition of the invention, the antigen is a viral antigen. In some embodiments of the invention the viral antigen is at least a strain of BEFV, BTV, or IBR. In some embodiments, saponin or Quil A is added to the viral antigen prior to adding SL-CD. In some embodiments the antigen may be live-attenuated, recombinant, killed or inactivated. In some embodiments, the antigen is live-attenuated. In some embodiments, the antigen is killed. In some embodiments, the antigen may be obtained from a frozen stock, dried stock, or a fresh stock. In some embodiments, the antigen is obtained from a frozen stock.

Immunological compositions of the invention may be prepared from viral cultures by methods that are standard in the art. For example, the virus may be propagated in tissue culture cells such as African green monkey kidney epithelial cells (Vero cells), human diploid fibroblasts, MDBK (Madin-Darby Bovine Kidney), or other bovine cells. The growth of the virus is monitored by standard techniques (observation of cytopathic effect, immunofluorescence or other antibody-based assays), and harvested when a sufficiently high viral titre has been achieved (such as 10⁶ TCID₅₀/mL). The viral stocks may be further concentrated or lyophilized by conventional methods before inclusion in the vaccine formulation. Other methods to prepare virus stock, such as those described by Thomas, et al. (1986, Agri-Practice, 7 (5):26-30), can be employed.

Immunological compositions of the invention can be given alone or as component of a polyvalent immunological composition, i.e., in combination with other immunological compositions. The virus in an immunogenic formulation can be live or killed; either live or killed virus can be lyophilized and, optionally, reconstituted as is known in the art. Immunogenic compositions can be provided in kits, which also can comprise appropriate labeling and instructions for administering an immunogenic composition to an animal subject (e.g., livestock, an ungulate, a companion animal) or a bird (e.g., poultry).

Immunogenic compositions comprising SL-CD and, saponin or Quil A; and at least one viral antigen also may comprise pharmaceutically and veterinary acceptable carriers. Such carriers are well known to those in the art and include, large, slowly metabolized macromolecules, such as proteins, polysaccharides, polylactic acids, polyglycolic acids, polymeric amino acids, amino acid copolymers, and inactive virus particles. Pharmaceutically and veterinary acceptable salts can also be used in the vaccine, for example, mineral salts such as hydrochlorides, hydrobromides, phosphates, or sulfates, as well as the salts of organic acids such as acetates, propionates, malonates, or benzoates. Vaccines also can contain liquids, such as water, saline, glycerol, and ethanol, as well as substances such as wetting agents, emulsifying agents, or pH buffering agents. Liposomes also can be used as carriers for killed virus. (See, for example, U.S. Pat. No. 5,422,120, PCT publication No. WO 95/13796, PCT publication No. WO 91/14445, or European Patent No. 524,968 B1.)

Immunogenic compositions of the present invention can be administered by intramuscular or subcutaneous routes, or by intranasal, intraperitoneal, intravenous, intradermal, intrabronchial, or oral routes. Immunogenic compositions of the invention can be administered by airspray, by eye inoculation, or by scarification. Another convenient method of delivering an immunogenic composition of the invention to mammals (such as livestock, ungulates, or companion animals) is by oral administration (e.g., in the feed or drinking water or in bait). It is particularly convenient to top-dress or mix feed with the immunogenic composition. Typically, large animals (e.g., livestock/ungulates such as cattle) are dosed with about 10⁶ TCID₅₀/mL may be 10^{6,5} to 10⁷, TCID₅₀ per dose of the immunogenic composition.

For single-dose administration, the immunogenic composition should contain an amount of BEFV corresponding to from about 10⁴ to about 10⁷ TCID₅₀/mL, preferably 10⁶ TCID₅₀/mL. About one to five mL of immunogenic composition, preferably 2 mL, may be administered per animal, intramuscularly, subcutaneously, or intraperitoneally.

The immunogenic composition should contain an amount of IBR corresponding to from about 6.8 logs/mL. About one to five mL of immunogenic composition comprising IBR, preferably 2 mL, may be administered per animal, intramuscularly, subcutaneously, or intraperitoneally.

The immunogenic composition should contain an amount of BTV corresponding from about 10^{6.7} TCID₅₀ of BTV serotype 1 and/or about 10^{7.3} TCID₅₀ of BTV serotype 8. About one to five mL of immunogenic composition comprising BTV, preferably 2 mL, may be administered per animal, intramuscularly, subcutaneously, or intraperitoneally.

The preparation of immunogenic compositions is found in the literature, for example in “Vaccine Design, The Subunit and Adjuvant Approach”, mentioned above, and “Vaccines” (2008, fifth edition, Plotkin, S. A. et al., editors, Saunders Elsevier).

The present invention provides immunological compositions that are particularly useful for the prophylaxis and treatment of BEF, IBR, or BTV infections in animals. Therefore, a further aspect of the present invention relates to methods for the prophylaxis and treatment of BEF, IBR, or BTV infections in animals characterized in that an immunogenic composition according to the present invention is administered to an animal in need of such prophylaxis or treatment. The immunogenic compositions of the present invention can be administered by intramuscular or subcutaneous injection or via intranasal, intratracheal, oral, cutane, percutane or intracutane administration. Preferably, for BEFV, IBR, or BTV vaccines, vaccination is subcutaneous or intramuscular, intramuscular being most preferred. Live vaccines for BEFV, IBR, or BTV are preferably administered from six months of age.

The invention also provides a method for the immunization of animals, in particular cattle, against one or various infectious agents simultaneously, that comprises the oral, nasal, subcutaneous, intradermal, intraperitoneal, intramuscular, or aerosol administration (or combinations thereof) of a vaccine that contains an immunologically effective amount of a composition provided by this invention.

### DEFINITIONS

The terms used herein have the meanings recognized and known to those of skill in the art, however, for convenience and completeness, particular terms and their meanings are set forth below.

As used in this specification and the appended claims, the singular forms “a”, “an”, and “the” include plural references unless the context clearly dictates otherwise. Thus, for example, references to “the method” include one or more methods, and/or steps of the type described herein and/or which will become apparent to those persons skilled in the art upon reading this disclosure and so forth.

The term “about” or “approximately” means within a statistically meaningful range of a value. Such a range can be within an order of magnitude, typically within 50%, more typically within 20%, more typically still within 10%, and even more typically within 5% of a given value or range. The allowable variation encompassed by the term “about” or “approximately” depends on the particular system under study, and can be readily appreciated by one of ordinary skill in the art.

An “infectious unit” of BEFV is defined as the amount of virus required for infecting or killing 50% of tissue culture cells. This may be expressed as the 50% tissue culture infective dose or TCID₅₀.

A virus is said to be attenuated if it has decreased virulence for the native host. A virus is considered inactivated if it is unable to propagate in a cell susceptible to infection by the virus.

The term “antigen” means a molecule that sometimes stimulates an immune response. An antigen is any substance that can be recognized by the adaptive immune system. Antigens are usually proteins or polysaccharides. An antigen may be a part of a bacterium, a virus, or other microorganism, such as a coat, capsule, cell-wall, flagella, fimbrae, or toxin. An antigen may also be a lipid or a nucleic acid. The antigen used in the composition may be obtained from a fresh culture, a frozen stock, a freeze-dried stock, or any other commonly available stock. If the antigen is a virus, it may be live-inactivated or attenuated.

“Adjuvant” means one or more substances that enhance the antigenicity of a composition, typically a vaccine composition. An adjuvant can serve as a tissue depot that slowly releases the antigen and also as a lymphoid system activator that non-specifically enhances the immune response (Hood, et al., Immunology, Second Ed., Menlo Park, Calif.: Benjamin/Cummings, 1984. p. 384). Often, a primary vaccination with an antigen alone, in the absence of an adjuvant, will fail to elicit a humoral or cellular immune response. Also, depending on the circumstances, a primary challenge with an antigen alone, in the absence of an adjuvant, may fail to elicit a sufficient humoral or cellular immune response. A number of cytokines or lymphokines have been shown to have immune-modulating activity, and thus are useful as adjuvants, including, the interleukins 1-α, 1-β, 2, 4, 5, 6, 7, 8, 10, 12 (see, e.g., U.S. Pat. No. 5,723,127), 13, 14, 15, 16, 17 and 18 (and its mutant forms); the interferons-α, β and γ; granulocyte-macrophage colony stimulating factor (GM-CSF) (see, e.g., U.S. Pat. No. 5,078,996); macrophage colony stimulating factor (M-CSF); granulocyte colony stimulating factor (G-CSF); and the tumor necrosis factors α and β. Still other adjuvants that are useful with the immunogenic compositions described herein include chemokines, including without limitation, monocyte chemotactic protein-1 (MCP-1), Macrophage Inflammatory Proteins (MIP) e.g., MIP-1α and MIP-1β, also known as CCL-3 and CCL-4; and Regulated on Activation Normal T cell Expressed and Secreted (RANTES); adhesion molecules, such as a selectin, e.g., L-selectin, P-selectin and E-selectin; mucin-like molecules, e.g., CD34 (also known as sialophorin, leukosialin, or SPN), GlyCAM-1 and MadCAM-1; a member of the integrin family such as lymphocyte function-associated molecules LFA-1, 2, and 3, VLA-1, Mac-1 and p150.95; a member of the immunoglobulin superfamily such as platelet endothelial cell adhesion molecule (PECAM), Intercellular adhesion molecules, e.g., ICAM-1, ICAM-2, ICAM-3, ICAM-4, and ICAM-5, CD2 and LFA-3; co-stimulatory molecules such as CD40 and CD40L; growth factors including vascular growth factor, nerve growth factor, fibroblast growth factor, epidermal growth factor, B7.2, PDGF, BL-1, and vascular endothelial growth factor; receptor molecules including Fas, TNF receptor, Flt, Apo-1, p55, WSL-1, DR3, TRAMP, Apo-3, AIR, LARD, NGRF, DR4, DR5, KILLER, TRAIL-R2, TRICK2, and DR6; and Caspase (ICE).

Suitable adjuvants used to enhance an immune response further include, without limitation, MPL™ (3-O-deacylated monophosphoryl lipid A, Corixa, Hamilton, Mont.), which is described in U.S. Pat. No. 4,912,094. Also suitable for use as adjuvants are synthetic lipid A analogs or aminoalkyl glucosamine phosphate compounds (AGP), or derivatives or analogs thereof, which are available from Corixa (Hamilton, Mont.), and which are described in U.S. Pat. No. 6,113,918. One such AGP is 2-[(R)-3-Tetradecanoyloxytetradecanoylamino]ethyl 2-Deoxy-4-O-phosphono-3-O—[(R)-3-tetradecanoyoxytetradecanoyl]-2-[(R)-3-tetradecanoyloxytetradecanoyl-amino]-b-D-glucopyranoside, which is also known as 529 (formerly known as RC529). This 529 adjuvant is formulated as an aqueous form (AF) or as a stable emulsion (SE).

Still other adjuvants include muramyl peptides, such as N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE); oil-in-water emulsions, such as MF59 (International PCT Publication No. WO 90/14837) (containing 5% Squalene, 0.5% Tween® 80, and 0.5% Span 85 (optionally containing various amounts of MTP-PE) formulated into submicron particles using a microfluidizer such as Model 110Y microfluidizer (Microfluidics, Newton, Mass.)), and SAF (containing 10% Squalene, 0.4% Tween 80, 5% pluronic-blocked polymer L121, and thr-MDP, either microfluidized into a submicron emulsion or vortexed to generate a larger particle size emulsion); incomplete Freund's adjuvant (IFA); aluminum salts (alum), such as aluminum hydroxide, aluminum phosphate, aluminum sulfate; Amphigen; Avridine; L121/squalene; D-lactide-polylactide/glycoside; pluronic polyols; killed Bordetella; saponins, such as Stimulon™ QS-21 (Antigenics, Framingham, Mass.), described in U.S. Pat. No. 5,057,540, ISCOMATRIX (CSL Limited, Parkville, Australia), described in U.S. Pat. No. 5,254,339, and immunostimulating complexes (ISCOMS); Mycobacterium tuberculosis; bacterial lipopolysaccharides; synthetic polynucleotides such as oligonucleotides containing a CpG motif (e.g., U.S. Pat. No. 6,207,646); IC-31 (Intercell AG, Vienna, Austria), described in European Patent Nos. 1,296,713 and 1,326,634; a pertussis toxin (PT) or mutant thereof, a cholera toxin or mutant thereof (e.g., International PCT Publication Nos. WO00/18434, WO02/098368 and WO02/098369); or an E. coli heat-labile toxin (LT), particularly LT-K63, LT-R72, PT-K9/G129; see, e.g., International PCT Publication Nos. WO 93/13302 and WO 92/19265.

Adjuvants that may be added to the compositions of the invention may include SL-CD, aluminum hydroxide, SP-oil, or carbopol, or a metabolizable oil such as one or more unsaturated terpene hydrocarbon(s), for example squalene or squalane, and a polyoxyethylene-polypropylene block copolymer such as Pluronic®

The term “Mammals” include monotremes (e.g., platypus), marsupials (e.g., kangaroo), and placentals, which include livestock (domestic animals raised for food, milk, or fiber such as hogs, sheep, cattle, and horses) and companion animals (e.g., dogs, cats). “Ungulates” include, but are not limited to, cattle (bovine animals), water buffalo, bison, sheep, swine, deer, elephants, and yaks. Each of these includes both adult and developing forms (e.g., calves, piglets, lambs, etc.). The immunogenic composition of the invention can be administered either to adults or developing mammals, preferably livestock.

The “immunologically effective amount” is the amount of antigen that will elicit an immune response. An immunologically effective amount of bovine ephemeral fever virus (BEFV) is the amount of BEFV that will elicit an immune response against bovine ephemeral fever virus. An immunologically effective amount of bovine herpesvirus 1 (IBR) is the amount of IBR that will elicit an immune response against IBR infection. An immunologically effective amount of blue tongue virus (BTV) is the amount of BTV that will elicit an immune response against BTV infection. The “immunologically effective amount” will depend on the species, breed, age, size, and health status of the recipient animal. The “immunologically effective amount” will be influenced by the previous exposure of the animal to one or more strain of the antigen whether that one or more strain is a virulent strain or an avirulent strain of virus. As used herein, an “immunologically effective amount” of bovine ephemeral fever virus (BEFV), when employed in combination with at least one suitable adjuvant, is that amount of BEFV that is sufficient to enhance the immunogenicity of the bovine ephemeral fever virus and thus provides for protective immunity against challenge with a virulent bovine ephemeral fever virus strain. In one embodiment, an immunologically effective amount of BEFV is about 10^{6.20} TCID₅₀ per mL composition.

As used herein, an “immunologically effective amount” of bovine herpesvirus 1 (IBR), when employed in combination with at least one suitable adjuvant, is that amount that is sufficient to enhance the immunogenicity of the bovine herpesvirus and thus provides for protective immunity against challenge with a virulent bovine herpesvirus strain. In one embodiment, an immunologically effective amount of IBR is about 6.8 logs per mL composition.

As used herein, an “immunologically effective amount” of blue tongue virus (BTV), when employed in combination with at least one suitable adjuvant, is that amount that is sufficient to enhance the immunogenicity of the blue tongue virus and thus provides for protective immunity against challenge with a virulent blue tongue virus strain. In one embodiment, an immunologically effective amount of BTV is about 10^{6.7} TCID₅₀ of BTV serotype 1 and/or about 10^{7.3} TCID₅₀ of BTV serotype 8 per mL composition.

In some embodiments of the invention, the viral antigen may be at least a strain of infectious bovine herpesvirus 1 (also referred to as bovine rhinotracheitis virus or IBR), parainfluenza virus, bovine respiratory syncytial virus, bovine viral diarrhea virus, foot and mouth disease virus, bluetongue virus, bovine ephemeral fever virus, canine parvovirus, canine distemper virus, canine adenovirus, canine parainfluenza virus, canine coronavirus, rabies virus, feline panleukopania virus, feline calicivirus, feline viral rhinotracheitis virus, feline infectious peritonitis virus, feline leukemia virus, feline immunodeficiency virus, West Nile virus, equine encephalomyelitis virus, equine influenza virus, equine herpes (rhinopneumonitis) virus, equine arteritis virus, porcine parvovirus, porcine cirocovirus, porcine reproductive and respiratory-syndrome virus, porcine rotavirus, swine influenza virus, pseudorabies virus, infectious bursal disease virus, Marek's disease virus, Newcastle disease virus, infectious bronchitis virus, infectious laryngotracheitis virus, avian encephalomyelitis virus, avian reovirus, avian influenza virus.

As used herein, the term “viral subunit” means a portion of a virion. For example, a bovine ephemeral fever virus (BEFV) subunit may be at least a portion of a BEFV virion, at least a portion of the BEFV genome, at least a portion of a BEFV-encoded protein, such as the BEFV nucleoprotein, the BEFV polymerase-associated protein, the BEFV matrix protein, the BEFV RNA-dependent RNA polymerase, or a BEFV glycoprotein.

As used herein, the term “immunogenic” means that the composition is capable of eliciting a humoral and/or cellular immune response. An immunogenic strain is also antigenic. An immunogenic composition is a composition that elicits a humoral and/or cellular immune response when administered to an animal.

The term “immunogenic composition” relates to any pharmaceutical composition containing an antigen, e.g. a microorganism, which composition can be used to elicit an immune response in an animal. The immune response can include a T cell response, a B cell response, or both a T cell and B cell response. The composition may serve to sensitize the mammal by the presentation of antigen in association with MHC molecules at the cell surface. In addition, antigen-specific T-lymphocytes or antibodies can be generated to allow for the future protection of an immunized host. An “immunogenic composition” may comprise a live, attenuated, or killed/inactivated antigen. The antigen may be a whole microorganism or an immunogenic portion derived therefrom that induces an immune response. The immunogenic composition may protect the animal from one or more symptoms associated with infection by the microorganism, or may protect the animal from death due to the infection with the microorganism.

The term “parenteral administration” as used herein means administration by some other means than through the gastrointestinal tract, particularly to the introduction of substances into an organism by intravenous, subcutaneous, intramuscular, or intramedullar injection, but also to other non-oral and non-nasal routes of administration such as intraperitoneal injection or topical application.

The terms “vaccine” or “vaccine composition”, are used interchangeably herein and refer to pharmaceutical compositions comprising at least one immunogenic composition that induces an immune response in an animal. A vaccine or vaccine composition may protect the animal from disease or possible death due to an infection, and may or may not include one or more additional components that enhance the immunological activity of the active component. A vaccine or vaccine composition may additionally comprise further components typical to pharmaceutical compositions. Further components may include, for example, one or more adjuvants or immunomodulators. The immunogenically active component of a vaccine may comprise complete live organisms in either their original form, or as attenuated organisms in a modified live vaccine, or organisms inactivated by appropriate methods in a killed or inactivated vaccine, or subunit vaccines comprising one or more immunogenic components of the virus, or genetically engineered, mutated or cloned vaccines prepared by methods known to those skilled in the art. A vaccine or vaccine composition may comprise one or simultaneously more than one of the elements described above.

Accordingly, in the present application, there may be employed conventional molecular biology, microbiology, and immunology techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y.

### EXAMPLES

### Example 1

### Preparation of Bovine Ephemeral Fever Virus/Quil A Mix

Live bovine ephemeral fever (BEF) viral antigen was obtained in frozen stock. After thawing at room temperature, the virus was combined with Quil A before adding the remaining ingredients for a vaccine.

Quil A powder, produced by Brenntag, was obtained from APS (A division of Nuplex Industries, Australia) as product code No. 04307503.

Quil A stock solution was produced by diluting in water to 10 mg/mL.

Briefly, 149.96 mL of live BEF antigen stock (1.38×10⁷ TCID₅₀/mL) was diluted with 36.34 mL of 9.643 g/mL NaCl and 20.70 mL of 10 mg/mL Quil A were added at 1 mg/mL. The mixture was stirred for 2.5 hours at room temperature.

### Example 2

### Preparation of BEFV Vaccine

To prepare a BEFV vaccine, the following ingredients were added, in order, while stirring the mixture for 5 minutes between additions.

384.3 mL 8.5 g/mL NaCl

94.87 g BEFV/QUIL A mixture prepared as in Example 1

120.00 mL of 10 mg/mL SL-CD* stock to obtain 20% (v/v)

1.36 g Thiomersal 9.9% (w/v)

*SL-CD was prepared as described by Hilgers et al. (Sulpholipo-cyclodextrin in squalene-in-water as a novel and safe vaccine adjuvant. Vaccine 17 (1999), pp 219-228.)

After all the ingredients were added, the vaccine was stirred for 30 minutes and the pH adjusted to 7.18.

The vaccine was stirred for an additional 30 minutes and used to fill labeled pillow packs.

### Example 3

### Animal Testing of BEFV Vaccine

Vaccine safety tests were carried out at the Fort Dodge Australia, Penrith site. Vaccine safety was tested in cattle in accordance with EP2002:0062 guidelines.

Ten Guinea pigs and two cattle were inoculated with the vaccine prepared above to determine the serological response to the BEF antigen fraction. The ten Guinea pigs weighing between 250 g and 400 g were each inoculated subcutaneously with 2.0 mL of vaccine. The Guinea pigs were bled six weeks after inoculation. Two cattle younger than 1 year were inoculated subcutaneously with 4 mL of vaccine. The cattle were bled on the 14 days after inoculation. The sera derived from these animals were tested by virus neutralization (VN) following the protocol of the Biosecurity Sciences Laboratory, Department of Primary Industries and Fisheries Animal Research Institute, Queensland (Australia). The virus neutralization test method used is in compliance with “Australia standard diagnostic procedures.”

The results of the trials for the two batches of vaccine are shown in Table 1, below:

TABLE 1
SAFETY RESULTS USING SINGLE DOSE BEF VACCINE

| | | RESULTS |
|---|---|---|
| TEST | SPECIFICATION | 20% SL-CD |
| Sterility | No bacterium or fungi | Pass |
| | detected | |
| Aqueous phase pH | 6.5-7.5 | 7.25 |
| BEF sera titre | N/A | Guinea pig = 32 |
| | | Cattle = Negative |
| Cattle Safety | No significant local or | No significant site reaction |
| | systemic reaction | post vaccination but |
| | | subsequent appearance of |
| | | lump at injection site |
| | | by 14 days |

Based on experiments using an inactivated BEF vaccine in cattle and rabbit trials, there is evidence for a correlation between laboratory animal serology and cattle protection from BEFV. Thus, the Guinea pig results shown in Table 1 confirm that the immunogenic composition of the invention will provide cattle protection from BEFV.

A single dose BEF vaccine formulation prepared as above produced a stable emulsion. This vaccine was tested for safety and initially gave no site-reactions in the cattle safety test. Although no systemic or behavioral reactions were noticed, the vaccine produced some local reactions at the injection site towards the end of the observation period.

Table 2, below, depicts the symptoms appearing in cattle after a single dose vaccination with 4 mL of the vaccine formulation listed above.

TABLE 2
SYSTEMIC AND LOCAL REACTION

| | |
|---|---|
| Day post | 054.6 (20% SLCD) |

| | inoculation | Cattle #1 | Cattle #2 |
|---|---|---|---|
| Systemic | 1 | Nil | Nil |
| reaction | 3 | Nil | Nil |
| | 5 | Nil | Nil |
| | 7 | Nil | Nil |
| | 10 | Nil | Nil |
| | 14 | Nil | Nil |
| Local (site) | 1 | 0 | 0 |
| reaction - cm | 3 | Not measured | Not measured |
| | 7 | 0 | 0 |
| | 14 | 2 × 9 | 2.5 × 13 |

| | |
|---|---|
| Any significant local or | No |
| systemic reaction? | |

In summary, use of an immunogenic composition comprising saponin (Quil A) and SL-CD as adjuvants in a formulation comprising BEFV produces an effective BEFV vaccine that is useful as a single dose vaccine.

### Example 4

### Preparation of IBR Vaccine Blends

To select a proper adjuvant for future vaccines, killed recombinant bovine herpesvirus-1, also known as infectious bovine rhinotracheitis virus (IBR) vaccine was prepared blended with different adjuvants and evaluated. Vaccines with three different adjuvant combinations were prepared with a 1× titer of 6.04 logs/mL of IBR(EU). Vaccine A contained AlOH (15%) & saponin; vaccine B contained 5% SP oil; and vaccine C contained 20% SL-CD & saponin.

TABLE 3
MONOVALENT rIBR (EU) VACCINE A WITH ALOH (15%) &
SAPONIN AS ADJUVANTS

| | | | | Total | |
|---|---|---|---|---|---|
| | | | | Volume | |
| Component | Stock conc factor | Amount/Dose | Conc./Dose | per 200 mL | |
| rIBR Lot # rIBREU-02 | 9.8X = 7.03 log₁₀/mL | 6.8 log₁₀s | 30.00% | 60.000 | mL |
| Sterile gel | 2%; with thimerosal | | 15.00% | 30.000 | mL |
| Saponin Solution (100 mg/mL) | 100 mg/mL | 1 mg | 0.50% | 1.000 | mL |
| 20% NZ Amine AS | 20% | NA | 5.00% | 10.000 | mL |
| 5% Thimerosal | 5% | NA | 0.19% | 0.370 | mL |
| Blending Diluent with | | | 49.32% | 98.630 | mL |
| Hepes w/o phenol red | | | | | |
| 20% HCl | | | | | mL |

TABLE 4
MONOVALENT rIBR (EU) VACCINE B WITH SP OIL AT 5%
AS ADJUVANT

| | | | | Total | |
|---|---|---|---|---|---|
| | | | | Volume | |
| Component | Stock conc factor | Amount/Dose | Conc./Dose | per 200 mL | |
| rIBR Lot # rIBREU-02 | 9.8X = 7.03 log₁₀/mL | 6.8 log₁₀s | 30.00% | 60.000 | mL |
| 20% NZ Amine AS | 20% | NA | 5.00% | 10.000 | mL |
| SP OIL | | 5% | 5.00% | 10.000 | mL |
| 5% Thimerosal | 5% | NA | 0.19% | 0.375 | mL |
| Blending Diluent w | | | 59.81% | 119.625 | mL |
| Hepes w/o phenol red | | | | | |
| 20% HCl | | | | | mL |

TABLE 5
MONOVALENT rIBR (EU) VACCINE C: WITH SL-CD AT 20% &
SAPONIN AS ADJUVANTS

| | | | | Total |
|---|---|---|---|---|
| | | | | Volume per |
| Component | Stock conc. factor | Amount/Dose | Conc./Dose | 200 mL |
| rIBR Lot # rIBREU-02 | 9.8X = 7.03 log₁₀/mL | 6.8 log₁₀s | 30.00% | 60.000 |
| SL-CD* | | | 20.00% | 40.000 |
| Saponin Solution | 100 mg/mL | 1 mg | 0.50% | 1.000 |
| (100 mg/mL) | | | | |
| 20% NZ Amine AS | 20% | NA | 5.00% | 10.000 |
| 5% Thimerosal | 5% | NA | 0.20% | 0.400 |
| Blending Diluent w | | | 44.30% | 88.600 |
| Hepes w/o phenol red | | | | |
| 20% HCl | | | | |

*SL-CD/squalane prepared as described by Hilgers et al. (Sulpholipo-cyclodextrin in squalene-in-water as a novel and safe vaccine adjuvant. Vaccine 17 (1999), pp219-228.)

### Example 5

### Animal Testing of IBR Vaccines

Vaccine tests were carried out in Iowa. A total of 27 calves, 5-6 months old, were randomized into groups as shown in Table 6, below:

TABLE 6
ANIMALS TESTED WITH IBR VACCINES

| Group | # Animals | Vaccine | # Vaccination |
|---|---|---|---|
| 1 | 5 | rIBR, Al(OH)₂/Saponin | 2 |
| 2 | 5 | rIBR, SP oil | 2 |
| 3 | 5 | rIBR, SL-CD/Saponin | 2 |
| 4 | 5 | rIBR, SL-CD/Saponin | 1 |
| 5 | 5 | None | None |
| 6 | 2 | None | None |

Except for calves from Groups 4-6, claves were vaccinated twice subcutaneously, 3 weeks apart. Two weeks after the second vaccination (or three weeks after vaccination for Group 4), all calves (except for two calves from Group 6) were challenged with virulent IBR virus intranasally. All calves were monitored daily for 14 days post challenge for the clinical signs of the disease. Clinical signs included but were not limited to mucopurulent nasal discharge, ocular discharge, dyspnea, poor appetite (off-feed), and depression. Rectal temperatures were also taken daily for 14 days post challenge. Animals were bled for serum periodically throughout the study and antibody against IBR was determined using serum neutralization assay. Nasal swabs were collected daily for virus isolation from two days prior to challenge through 14 days post challenge. The virus titer isolated from each calf for each day was determined. One animal from Group 2 was removed from the study prior to the challenge due to a poor health.

The observed clinical signs that were associated with IBR challenge are summarized in Table 7, below, and the viral shedding results are depicted in Table 8. The Anti-IBR serum neutralization antibody titers are listed in Table 9.

TABLE 7
MEAN INCIDENCES^{a} OF CLINICAL SIGNS OBSERVED IN
ANIMALS CHALLENGED WITH VIRULENT IBR

| | | Mucopurulent | |
|---|---|---|---|
| Group | Fever^{b} | Nasal Discharge | Coughing |
| rIBR, Al(OH)₂/Saponin | 1.8 ± 1.5 | 0.4 ± 0.5 | 1.2 ± 0.8 |
| rIBR, SP oil | 3.0 ± 2.9 | 0.5 ± 0.6 | 1.0 ± 2.0 |
| rIBR, SL-CD/Saponin, two | 2.8 ± 3.4 | 0.6 ± 0.9 | 0.6 ± 0.9 |
| doses | | | |
| rIBR, SL-CD/Saponin, one | 4.0 ± 3.2 | 0.8 ± 0.8 | 0.6 ± 0.5 |
| dose | | | |
| Challenge Control | 5.2 ± 1.8 | 1.0 ± 1.0 | 2.2 ± 3.3 |
| Environmental Control | 0 | 0 | 0 |

^{a}The value is expressed as mean ± standard deviation.
^{b}Rectal temperature ≧103.5° F. and 1° F. above baseline.

Due to the small group size, the differences observed between the vaccinated animals and the controls were not statistically different. However, the numerical differences do indicate a vaccination effect, especially for the first three groups.

The incidence and titer of viral shedding are depicted in Table 8, below:

TABLE 8
MEAN VIRAL SHEDDING TITERS^{a} AND INCIDENCES^{b}
IN ANIMALS CHALLENGED WITH VIRULENT IBR

| Group | Titer | Incidence |
|---|---|---|
| rIBR, Al(OH)₂/Saponin | 6.1 ± 0.6* | 7.2 ± 1.5* |
| rIBR, SP oil | 5.9 ± 0.8* | 7.3 ± 0.5* |
| rIBR, SL-CD/Saponin, two doses | 4.2 ± 2.1 | 4.6 ± 2.7 |
| rIBR, SL-CD/Saponin, one dose | 6.1 ± 0.5* | 6.2 ± 1.1* |
| Challenge Control | 6.6 ± 0.4* | 8.2 ± 1.8* |
| Environmental Control | 0 | 0 |

^{a}The value is expressed as mean log₁₀ TCID₅₀ titer ± standard deviation.
^{b}The value is expressed as mean ± standard deviation.
*The indicated value is significantly different from that of the group vaccinated with two doses of rIBR with SL-CD/Saponin adjuvant, p < 0.05.

Results from the viral shedding indicate that SL-CD+Saponin provided the best protection by reducing the numbers (at least 100 folds) and incidences of virus shed. This demonstrated protection is likely due to a significantly higher antibody titer at the time of experimental challenge as shown in Table 9, below:

TABLE 9
ANTI-IBR SERUM NEUTRALIZATION ANTIBODY TITERS^{a}
IN ANIMALS VACCINATED WITH A KILLED rIBR
VACCINE IN DIFFERENT ADJUVANTS

| Group | Titer |
|---|---|
| rIBR, Al(OH)₂/Saponin | 25 ± 2* |
| rIBR, SP oil | 32 ± 2* |
| rIBR, SL-CD/Saponin, two doses | 63 ± 4 |
| rIBR, SL-CD/Saponin, one dose | 4 ± 3* |

^{a}The values are expressed as geometric mean titer ± standard deviation. Serum samples were collected at the day of challenge.
*The indicated value is significantly different from that of the group vaccinated with two doses of rIBR with SL-CD/Saponin adjuvant, p < 0.05.

Results from this study indicate that, among the three adjuvants evaluated, the SL-CD/Saponin combination provides the best performance.

### Example 6

### Preparation of Bluetongue Virus Vaccines

Five different inactivated vaccines against bluetongue virus serotype 1 and 8 were formulated with different BTV8 antigen concentration and with different adjuvant composition. The titer of BTV serotype 1 (10^{6.7} TCID₅₀) remained constant in all the vaccines tested. Calves received two inoculations two weeks apart. The composition of vaccines E-43, E-44, E-45, E-47, and E-48 are depicted in Tables 10 through 14, below:

TABLE 10
COMPOSITION OF BTV VACCINE E-43*

| COMPONENT | AMOUNT |
|---|---|
| BTV inactivated serotype 1, strain ALG2006/01 E1 | 10^{6.7} TCID₅₀ |
| BTV inactivated serotype 8, Strain BEL2006/02 | 10^{7.3} TCID₅₀ |
| Aluminium hydroxide gel 3% | 4 mg Al³⁺ |
| Saponin | 0.4 mg |
| Saline Solution | q.s. 2.0 mL |
| Thiomersal | 0.2 mg |

*Batch E-43 of Zulvac ® 1 + 8 Bovis vaccine is the one registered in Spain (Emergency License).

TABLE 11
COMPOSITION OF BTV VACCINE E-44

| COMPONENT | AMOUNT |
|---|---|
| BTV inactivated serotype 1, strain ALG2006/01 E1 | 10^{6.7} TCID₅₀ |
| BTV inactivated serotype 8, Strain BEL2006/02 | 10^{7.5} TCID₅₀ |
| Aluminium hydroxide gel 3% | 4 mg Al³⁺ |
| Saponin | 0.4 mg |
| Saline Solution | q.s. 2.0 mL |
| Thiomersal | 0.2 mg |

TABLE 12
COMPOSITION OF BTV VACCINE E-45

| COMPONENT | AMOUNT |
|---|---|
| BTV inactivated serotype 1, strain ALG2006/01 E1 | 10^{6.7} TCID₅₀ |
| BTV inactivated serotype 8, Strain BEL2006/02 | 10^{7.3} TCID₅₀ |
| Aluminium hydroxide gel 3% | 4 mg Al³⁺ |
| Saponin | 1.0 mg |
| Saline Solution | q.s. 2.0 mL |
| Thiomersal | 0.2 mg |

TABLE 13
COMPOSITION OF BTV VACCINE E-47

| COMPONENT | AMOUNT |
|---|---|
| BTV inactivated serotype 1, strain ALG2006/01 E1 | 10^{6.7} TCID₅₀ |
| BTV inactivated serotype 8, Strain BEL2006/02 | 10^{7.3} TCID₅₀ |
| SL-CD* | 20% |
| Saponin | 1.0 mg |
| Saline Solution | q.s. 2.0 mL |
| Thiomersal | 0.2 mg |

*SL-CD/squalane prepared as described by Hilgers et al. (Supra).

TABLE 14
COMPOSITION OF BTV VACCINE E-48

| COMPONENT | AMOUNT |
|---|---|
| BTV inactivated serotype 1, strain ALG2006/01 E1 | 10 ^{6.7} TCID₅₀ |
| BTV inactivated serotype 8, Strain BEL2006/02 | 10^{7.0} TCID₅₀ |
| SLCD | 20% |
| Saponin | 1.0 mg |
| Saline Solution | q.s. 2.0 mL |
| Thiomersal | 0.2 mg |

### Example 7

### Seroneutralization Results

Neutralizing antibody titers were measured in all calves at one week (+28) and at two weeks (+35) post-vaccination. The seroneutralization results are shown in tables 15 through 20, below. Presence of neutralizing antibodies is indicative of protection but animals without neutralizing antibodies can be also protected due to cell-mediated response.

TABLE 15
RESULTS OBTAINED WITH VACCINE E-43
BTV-1 = 10 exp^{6,7} BTV-8 = 10 exp^{7,3}

| | |
|---|---|
| +28 | +35 |

| Calf | BTV 1 | BTV 8 | BTV 1 | BTV 8 |
|---|---|---|---|---|
| 79 | 1.4 | 1.4 | 4 | 4 |
| 243 | 1 | 2 | 16 | 5.7 |
| 349 | 4 | 2.8 | 32 | 11.3 |
| 365 | 2 | 1 | 16 | 2.8 |
| 524 | 1.4 | 5.7 | 22.6 | 4 |
| 638 | 1.4 | 2.8 | 22.6 | 22.6 |
| 660 | 11.3 | 1 | 22.6 | 5.7 |
| 695 | 8 | 2.8 | 22.6 | 8 |
| 720 | 4 | 1.4 | 16 | 4 |
| 841 | 8 | 1.4 | 32 | 5.7 |
| 893 | 16 | 11.3 | 22.6 | 16 |
| 922 | 32 | 4 | 64 | 32 |
| 1027 | 11.3 | 4 | 45.3 | 11.3 |
| 1324 | 8 | 8 | 45.3 | 11.3 |
| 1327 | 8 | 4 | 64 | 22.6 |
| 1608 | 45.3 | 11.3 | 45.3 | 8 |
| 2039 | 11.3 | 16 | 128 | 16 |
| 2996 | 2 | 5.7 | 4 | 5.7 |
| 5371 | 5.7 | 8 | 8 | 8 |
| 6352 | 2.8 | 1.4 | 16 | 16 |
| GM | 5.4 | 3.4 | 23.4 | 8.9 |

TABLE 16
RESULTS OBTAINED WITH VACCINE E-44

| | |
|---|---|
| +28 | +35 |

| Calf | BTV 1 | BTV 8 | BTV 1 | BTV 8 |
|---|---|---|---|---|
| 287 | 2 | 1.4 | 4 | 2 |
| 332 | 22.6 | 8 | 128 | 11.3 |
| 346 | 45.3 | 32 | 32 | 16 |
| 502 | 8 | 2.8 | 16 | 16 |
| 539 | 11.3 | 4 | 90.5 | 8 |
| 608 | 4 | 1.4 | 16 | 1 |
| 628 | 16 | 2 | 32 | 16 |
| 630 | 2.8 | 1.4 | 22.6 | 1.4 |
| 647 | 45.3 | 16 | 128 | 32 |
| 887 | 22.6 | 2.8 | 128 | 5.7 |
| 1046 | 11.3 | 2 | 45.3 | 1.4 |
| 2881 | 45.3 | 4 | 16 | 32 |
| 3035 | 16 | 8 | 22.6 | 11.3 |
| 3046 | 16 | 5.7 | 16 | 8 |
| 3824 | 2.8 | 2 | 11.3 | 1 |
| 4341 | 22.6 | 4 | 11.3 | 4 |
| 4656 | 22.6 | 5.7 | 22.6 | 8 |
| 6927 | 8 | 1 | 8 | 4 |
| 6997 | 5.7 | 8 | 22.6 | 2 |
| 8097 | 1 | 5.7 | 2.8 | 5.7 |
| GM | 10.5 | 3.9 | 23.0 | 5.7 |

TABLE 17
RESULTS OBTAINED WITH VACCINE E-45

| | |
|---|---|
| +28 | +35 |

| Calf | BTV 1 | BTV 8 | BTV 1 | BTV 8 |
|---|---|---|---|---|
| 87 | 8 | 2 | 16 | 8 |
| 296 | 4 | 4 | 11.3 | 5.7 |
| 520 | 11.3 | 11.3 | 64 | 11.3 |
| 607 | 8 | 8 | 90.5 | 11.3 |
| 612 | 11.3 | 16 | 64 | 16 |
| 623 | 11.3 | 8 | 8 | 11.3 |
| 717 | 16 | 4 | 45.3 | 4 |
| 731 | 16 | 16 | 128 | 16 |
| 789 | 8 | 11.3 | 16 | 8 |
| 871 | 64 | 8 | 90.5 | 16 |
| 886 | 11.3 | 2.8 | 128 | 5.7 |
| 914 | 4 | 1 | 64 | 2 |
| 977 | 11.3 | 8 | 45.3 | 32 |
| 1024 | 32 | 8 | 90.5 | 32 |
| 1542 | 4 | 1.4 | 22.6 | 5.7 |
| 2654 | 4 | 2 | 16 | 5.7 |
| 2843 | 16 | 5.7 | 45.3 | 16 |
| 6772 | 11.3 | 5.7 | 45.3 | 4 |
| 8004 | 5.7 | 4 | 8 | 8 |
| 8327 | 4 | 4 | 22.6 | 32 |
| GM | 9.7 | 5.1 | 36.8 | 9.7 |

TABLE 18
RESULST OBTAINED WITH VACCINE E-47

| | |
|---|---|
| +28 | +35 |

| Calf | BTV 1 | BTV 8 | BTV 1 | BTV 8 |
|---|---|---|---|---|
| 22 | 2 | 1.4 | 4 | 16 |
| 345 | 32 | 8 | 181 | 22.6 |
| 422 | 2.8 | 5.7 | 4 | 8 |
| 501 | 8 | 4 | 32 | 8 |
| 535 | 8 | 16 | 22.6 | 8 |
| 536 | 4 | 1 | 5.7 | 1.4 |
| 551 | 1 | 2 | 11.3 | 8 |
| 617 | 8 | 8 | 32 | 16 |
| 735 | 1 | 1 | 16 | 2 |
| 748 | 16 | 4 | 64 | 16 |
| 817 | 4 | 2.8 | 11.3 | 16 |
| 894 | 4 | 4 | 16 | 4 |
| 982 | 5.7 | 2 | 16 | 16 |
| 1009 | 1 | 4 | 11.3 | 11.3 |
| 1157 | 16 | 8 | 128 | 22.6 |
| 1187 | 5.7 | 8 | 45.3 | 11.3 |
| 5515 | 8 | 2.8 | 64 | 4 |
| 5982 | 5.7 | 2.8 | 11.3 | 2 |
| 6776 | 16 | 8 | 22.6 | 11.3 |
| 7797 | 4 | 1 | 32 | 8 |
| GM | 5.1 | 3.5 | 21.5 | 8.3 |

TABLE 19
RESULTS OBTAINED WITH VACCINE E-48

| | |
|---|---|
| +28 | +35 |

| Calf | BTV 1 | BTV 8 | BTV 1 | BTV 8 |
|---|---|---|---|---|
| 130 | 4.0 | 2 | 45.3 | 2 |
| 316 | 5.7 | 2 | 16 | 4 |
| 344 | 2.8 | 1 | 16 | 2.8 |
| 428 | 5.7 | 2.8 | 8 | 2.8 |
| 442 | 1.4 | 2.8 | 16 | 1 |
| 514 | 22.6 | 2.8 | 64 | 8 |
| 645 | 64.0 | 5.7 | 128 | 4 |
| 740 | 32.0 | 5.7 | 181 | 5.7 |
| 787 | 1.0 | 2.8 | 45.3 | 8 |
| 790 | 5.7 | 2 | 32 | 1 |
| 836 | 2.8 | 1 | | |
| 890 | 8.0 | 2.8 | 32 | 5.7 |
| 1278 | 16.0 | 4 | 32 | 8 |
| 1789 | 11.3 | 4 | 90.5 | 16 |
| 3654 | 1.4 | 1.4 | 1.4 | 4 |
| 6783 | 4.0 | 1 | 16 | 1.4 |
| 7085 | 32.0 | 4 | 22.6 | 4 |
| 9872 | 16.0 | 1.4 | 32 | 4 |
| 40552 | 5.7 | 2.8 | 8 | 1 |
| 80552 | 8 | 1.4 | 5.7 | 2 |
| GM | 7.0 | 2.3 | 24.3 | 3.4 |

TABLE 20
CONTROLS

| | |
|---|---|
| +28 | +35 |

| Calf | BTV 1 | BTV 8 | BTV 1 | BTV 8 |
|---|---|---|---|---|
| 295 | 1 | 1 | 1 | 1 |
| 377 | 1 | 1 | 1 | 1 |
| 537 | 1 | 1 | 1 | 1 |
| 548 | 1 | 1 | 1 | 1 |
| 643 | 1 | 1 | 1 | 1 |
| 659 | 1 | 1 | 1 | 1 |
| 671 | 1 | 1 | 1 | 1 |
| 679 | 1 | 1 | 1 | 1 |
| 724 | 1 | 1 | 1 | 1 |
| 733 | 1 | 1 | 1 | 1 |
| 778 | 1 | 1 | 1 | 1 |
| 1079 | 1 | 1 | 1 | 1 |
| 1221 | 1 | 1 | 1 | 1 |
| 1791 | 1 | 1 | 1 | 1 |
| 2117 | 1 | 1 | 1 | 1 |
| 2568 | 1 | 1 | 1 | 1 |
| 3659 | 1 | 1 | 1 | 1 |
| 3931 | 1 | 1 | 1 | 1 |
| 5523 | 1 | 1 | 1 | 1 |
| 8287 | 1 | 1 | 1 | 1 |
| GM | 1.0 | | 1.0 | 1.0 |

The presence of viremia for BTV1 and BTV8 was determined at 4, 5, and 8 days post challenge in animals vaccinated with Vaccines E-43: ZULVAC 1+8 (BTV1: 106.7+BTV8: 107.3) (Al3++Saponin: actual formulation) and E-47: ZULVAC 1+8 (BTV1 106.7+BTV8: 107.3) (SLCD+2.5× Saponin: new adjuvant).

Vaccine E-43: ZULVAC 1+8 (BTV1: 10^{6.7}+BTV8: 10^{7.3}) (Al³⁺+Saponin: actual formulation)
100% prevention of viremia for BTV1 (0/8)
87.5% prevention of viremia for BTV8 (1/8)

Vaccine E-44: ZULVAC 1+8 (BTV1 10^{6.7}+BTV8: 10^{7.5}) (Al³⁺+Saponin 1.58 more antigen of BTV8 than the actual formulation)
100% prevention of viremia for BTV1 (0/8)
87.5% prevention of viremia for BTV8 (1/8)

The results directly above indicate that an increase of BTV serotype 8 in a vaccine by 1.58× does not induce a better protection.

Vaccine E-47: ZULVAC 1+8 (BTV1 10^{6.7}+BTV8: 10^{7.3}) (SLCD+2.5× Saponin: new adjuvant)
100% prevention of viremia for BTV1 (0/8)
100% prevention of viremia for BTV8 (0/8)

Vaccine E-45: ZULVAC 1+8 (BTV1 106.7+BTV8: 107.3) (Al3++2× Saponin: 2× more Saponin than the actual formulation)
100% prevention of viremia for BTV1 (0/8)
100% prevention of viremia for BTV8 (0/8)

### Example 8

### Production of Gamma Interferon

The Bovigam TB test (Prionics) was used for the detection of gamma interferon in blood samples. Briefly, peripheral blood mononuclear cells (PBMC) were prepared, and were stimulated, part with VP7 and part with VP2. Production of gamma interferon was detected only after stimulation of the blood cells with VP7.

The results of evaluation of the specific production of γ-IFN in animals vaccinated with vaccines E-43 and E-47 follows. 1^{st} Vaccination (D+0); 2^{nd} Vaccination (D+21); Challenge (D+45)

A total of 30, 3 months-old Frisean calves without antibodies against BTV were included in the study. The sex of the calves was not taken into account. Only normal and healthy animals were included in the study. Their health condition was verified upon arrival. The animals were individually identified with ear tags. The 30 seronegative Frisean calves were randomly allocated into four treatment groups (using Microsoft Excel program), as follows:

Group 1: 10 calves, vaccinated and revaccinated with vaccine E-43

Group 2: 10 calves, vaccinated and revaccinated with vaccine E-47

Group 3: 10 control calves, non vaccinated

Vaccinations were performed by intramuscular route (i.m.), the most common route for vaccine administration in cattle, using 2 mL of vaccine.

Calves in groups 1 and 2 were vaccinated at day 0 (D.0) and revaccinated 3 weeks later.

Calves in group 3 were left as non vaccinated controls.

Blood was taken from the calves at day 0 (D.0), before the first vaccination; before the revaccination (or 2^{nd} vaccination) three weeks later (D+21); and at day 42, before challenge (D+42). Peripheral blood mononuclear cells (PBMCs) were prepared from the individual samples.

Twenty-four days after the 2^{nd} vaccination, the calves were moved to Fort Dodge Veterinaria's Challenge Facilities No. 3. where 24 days after revaccination (D+45), 8 animals of each group were challenged with BTV-1 or BTV-8. Blood was taken from the animals 5 days post challenge, for the evaluation of specific production of γ-IFN against VP7 and VP2.

### γ-IFN Detection

Blood collected in the presence of heparin was taken from all animals in the experiment at the day of each vaccination, the day before challenge and 5 days post-infection. PBMC were extracted on a density gradient (Histopaque 1077), washed and resuspended to a final concentration of 5×10⁶ cells/mL in RPMI 1640 medium supplemented with fetal calf serum. Cells were plated in 96 well plates with either recombinant proteins VP2 and VP7 (1 μg/mL). Concavalin A (5 μg/mL) was used as a positive control. Plates were incubated at 37° C. for 16-overnight. γ-IFN assays were performed in the supernatants using the bovine interferon test (Bovigam TB, Prionics). The results were expressed in A450 units after subtraction of the non-stimulated values of each animal.

Only VP7 recombinant protein was able to induce specific production of γ-IFN in vaccinated animals after a second vaccination.

On the day of challenge, 3 out of 10 (30%) calves vaccinated with vaccine E-47 showed production of γ-IFN against VP7. Five days after challenge, the proportion of positive animals increased to 63%. Animals vaccinated with vaccine E-43 showed positive production of γ-IFN against VP7 five days after challenge (2 out of 8, 25%) but not the day of challenge.

Results are shown in Table 21 expressed as A450 units and as proportions of positive production of γ-IFN (>0.065).

TABLE 21
STATISTICS OF EACH TREATMENT GROUP THE DAY OF EACH VACCINATION,
THE DAY OF CHALLENGE AND 5 DAYS POST-CHALLENGE

| Treatment | | VP2_D0V1 | VP7_D0V1 | VP2_D0V2 | VP7_D0V2 | VP2D0Ch | VP7D0Ch | VP2D5Ch | VP7D5Ch |
|---|---|---|---|---|---|---|---|---|---|
| E-43 | Mean | .00180 | .00060 | .00156 | .00600 | .00130 | .00980 | .00200 | .04400 |
| | N | 10 | 10 | 9 | 9 | 10 | 10 | 8 | 8 |
| | Std. Deviation | .004467 | .001265 | .002555 | .010235 | .002406 | .020596 | .002976 | .078258 |
| | Minimum | .000 | .000 | .000 | .000 | .000 | .000 | .000 | .000 |
| | Maximum | .014 | .003 | .007 | .029 | .007 | .068 | .007 | .207 |
| | Median | .00000 | .00000 | .00000 | .00000 | .00000 | .00300 | .00000 | .00550 |
| E-47 | Mean | .00300 | .00100 | .00911 | .00489 | .00220 | .13830 | .00800 | .35600 |
| | N | 10 | 10 | 9 | 9 | 10 | 10 | 8 | 8 |
| | Std. Deviation | .009487 | .003162 | .012354 | .007061 | .002658 | .267033 | .015501 | .356680 |
| | Minimum | .000 | .000 | .000 | .000 | .000 | .001 | .000 | .003 |
| | Maximum | .030 | .010 | .033 | .020 | .008 | .816 | .045 | .831 |
| | Median | .00000 | .00000 | .00400 | .00200 | .00150 | .00550 | .00150 | .30800 |
| Control | Mean | .00520 | .00320 | .00322 | .01189 | .00310 | .00160 | .00233 | .00944 |
| | N | 10 | 10 | 9 | 9 | 10 | 10 | 9 | 9 |
| | Std. Deviation | .011124 | .007315 | .006320 | .023385 | .005195 | .002221 | .004000 | .011727 |
| | Minimum | .000 | .000 | .000 | .000 | .000 | .000 | .000 | .000 |
| | Maximum | .030 | .022 | .019 | .069 | .017 | .007 | .012 | .031 |
| | Median | .00000 | .00000 | .00000 | .00000 | .00150 | .00100 | .00000 | .00400 |

The day of challenge, when using the Mann-Whitney test, significant differences were found between the groups vaccinated with E-43 and E-47 and the control group: E-43 versus controls: p = 0.035; E-47 versus controls: P = 0.003
Five days after challenge, when using the Mann-Whitney test, significant differences were found between the groups vaccinated with E-43 and E-47 and the control group: E-43 versus E-47: p = 0.021; E-47 versus controls: p = 0.006

TABLE 22
CROSSTAB BETWEEN THE TWO VACCINES
ON THE DAY OF CHALLENGE

| | | | Treatment | | |
|---|---|---|---|---|---|
| | | | E-43 | E-47 | Total |
| VP7D + | Negative | Count | 10 | 7 | 17 |
| 0Ch | | % within Treatment | 100.0% | 70.0% | 85.0% |
| | Positive | Count | 0 | 3 | 3 |
| | | % within Treatment | .0% | 30.0% | 15.0% |
| | Total | Count | 10 | 10 | 20 |
| | | % within Treatment | 100.0% | 100.0% | 100.0% |

No significant differences: p = 0.105

TABLE 23
CROSSTAB BETWEEN THE TWO VACCINES
5 DAYS AFTER CHALLENGE

| | | | Treatment | | |
|---|---|---|---|---|---|
| | | | E-43 | E-47 | Total |
| VP7D + | Negative | Count | 6 | 3 | 9 |
| 5Ch | | % within Treatment | 75.0% | 37.5% | 56.3% |
| | | Count | 2 | 5 | 7 |
| | Positive | % within Treatment | 25.0% | 62.5% | 43.8% |
| | Total | Count | 8 | 8 | 16 |
| | | % within Treatment | 100.0% | 100.0% | 100.0% |

No significant differences: p = 0.157

TABLE 24
CROSSTAB BETWEEN VACCINE E-47 AND CONTROLS
ON THE DAY OF CHALLENGE

| | | | Treatment | | |
|---|---|---|---|---|---|
| | | | E-47 | Control | Total |
| VP7D + 0Ch | Negative | Count | 7 | 10 | 17 |
| | | % within | 70.0% | 100.0% | 85.0% |
| | | Treatment | | | |
| | Positive | Count | 3 | 0 | 3 |
| | | % within | 30.0% | .0% | 15.0% |
| | | Treatment | | | |
| | Total | Count | 10 | 10 | 20 |
| | | % within | 100.0% | 100.0% | 100.0% |
| | | Treatment | | | |

No significant differences: p = 0.105

TABLE 25
CROSSTAB BETWEEN VACCINE E-47 AND CONTROLS
5 DAYS AFTER CHALLENGE

| | | | Treatment | | |
|---|---|---|---|---|---|
| | | | E-47 | Control | Total |
| VP7D + 5Ch | Negative | Count | 3 | 9 | 12 |
| | | % within | 37.5% | 100.0% | 70.6% |
| | | Treatment | | | |
| | Positive | Count | 5 | 0 | 5 |
| | | % within | 62.5% | .0% | 29.4% |
| | | Treatment | | | |
| | Total | Count | 8 | 9 | 17 |
| | | % within | 100.0% | 100.0% | 100.0% |
| | | Treatment | | | |

Significant differences: p = 0.009

TABLE 26
CROSSTAB BETWEEN VACCINE E-43 AND CONTROLS
5 DAYS AFTER CHALLENGE

| | | | Treatment | | |
|---|---|---|---|---|---|
| | | | E-43 | Control | Total |
| VP7D + 5Ch | Negative | Count | 6 | 9 | 15 |
| | | % within | 75.0% | 100.0% | 88.2% |
| | | Treatment | | | |
| | Positive | Count | 2 | 0 | 2 |
| | | % within | 25.0% | .0% | 11.8% |
| | | Treatment | | | |
| | Total | Count | 8 | 9 | 17 |
| | | % within | 100.0% | 100.0% | 100.0% |
| | | Treatment | | | |

### CONCLUSION

Calves vaccinated with ZULVAC 1+8, batch E-47 (BTV1: 10^{6.7}+BTV8: 10^{7.3}) (SLCD+2.5× Saponin: new adjuvant) showed higher amount of γ-IFN the day of challenge (3 weeks after 2^{nd} vaccination) and 5 days post-challenge than controls and than calves vaccinated with ZULVAC 1+8 (BTV1: 10^{6.7}+BTV8: 10^{7.3}) (Al³⁺+Saponin: actual formulation).
Calves vaccinated with vaccine E-43 (actual formulation) did not showed production of γ-IFN against VP7 until 5 days after challenge. Its values were significant lower (p=0.021) that ones induced by vaccine E-47 (new adjuvant).
Only VP7 recombinant protein has been able to induce detectable amounts of γ-IFN, therefore VP7 could be an enhancer of cellular immunity.

What is claimed:

1. An immunological composition comprising sulpholipo-cyclodextrin (SL-CD) and saponin.

2. The immunological composition of claim 1 further comprising at least one antigen.

3. The immunological composition of claim 2, wherein the at least one antigen is selected from bacteria, viruses, peptides, polypeptides, nucleic acids, or combinations thereof.

4. The immunological composition of claim 3, wherein the at least one antigen is a veterinary antigen.

5. The immunological composition of claim 4, wherein the at least one antigen is a bovine antigen.

6. The immunological composition of claim 3, wherein the at least one antigen is a viral antigen.

7. The immunological composition of claim 6, wherein the viral antigen is bovine ephemeral fever virus (BEFV); bovine herpes virus 1 (IBR), or bluetongue virus (BTV).

8. The immunological composition of claim 7 wherein the SL-CD is present at a final concentration of about 0.2 mL/mL.

9. The immunological composition of claims 7 wherein the saponin is present at a final concentration of about 0.5 mg/mL.

10. The immunological composition of claim 7 wherein the saponin is Quil A.

11. The immunological composition of claim 10 wherein the Quil A is present at a final concentration of about 0.1 mg/mL to about 0.2 mg/mL.

12. The immunological composition of claim 11 wherein the Quil A is present at a final concentration of about 0.158 mg/mL.

13. A method for eliciting an immune response in an animal in need thereof, which comprises administering to the animal the immunological composition of claim 8 wherein the saponin is present at a final concentration of about 0.5 mg/mL, or wherein the saponin is Quil A, and the Quil A is present at a final concentration of about 0.1 mg/mL to about 0.2 mg/mL, or the Quil A is present at a final concentration of about 0.158 mg/mL.

14. An immunological composition prepared by combining Quil A and a viral antigen prior to adding at least one additional adjuvant.

15. The immunological composition prepared as in claim 14, further comprising at least one additional adjuvant.

16. The immunological composition prepared as in claim 15, wherein the at least one additional adjuvant is selected from SL-CD, aluminum hydroxide, SP-oil, or carbopol.

17. The immunological composition prepared as in claim 16, wherein the at least one additional adjuvant is SL-CD.

18. The immunological composition prepared as in claim 17 wherein the SL-CD is present at a final concentration of about 0.2 mL/mL.

19. The immunological composition prepared as in claim 14 wherein the Quil A is present at a final concentration of about 0.1 mg/mL to about 0.2 mg/mL.

20. The immunological composition prepared as in claims 14 wherein the Quil A is present at a final concentration of about 0.158 mg/mL.

21. The immunological composition prepared as in claim 14 wherein the viral antigen is a bovine antigen.

22. The immunological composition prepared as in claim 21 wherein the viral antigen is BEFV, IBR, or BTV.

23. A method for eliciting an immune response against BEFV in an animal which comprises administering to the animal the composition according to claim 22.

24. The method of claim 23 wherein the immune response is elicited after administration of a single dose of the composition.

25. The method of claim 23 wherein the immune response is a protective immune response.

26. A kit for eliciting an immune response in an animal, comprising SL-CD and saponin, or SL-CD and Quil A

27. The kit of claim 26, further comprising at least one antigen.

28. The kit of claim 27, wherein the at least one antigen is selected from bacteria, viruses, peptides, polypeptides, nucleic acids, or combinations thereof.

29. The kit of claim 28, wherein the at least one antigen is a viral antigen.

30. The kit of claim 29, wherein the viral antigen is selected from BEFV, IBR, and BTV.
